(19) United States
(12) Patent Application Publication
(10) Pub. No.: US 2026/0356876 A1
(43) Pub. Date: Nov. 12, 2026

(54) Methods for the Synthesis of RNA Molecules Having a 5’-CAP and/or A 3’-POLY(A) Tail Comprising Deuterium, and Stabilised RNA Molecules Obtained Therefrom

(71) Applicant: deutraMed Solutions Ltd, Collingwood (CA)

(72) Inventors: Pavel Gris, Markdale (CA); Alfred James Farmilo, Belleville (CA)

(73) Assignee: deutraMed Solutions Ltd, Collingwood, ON (CA)

(21) Appl. No.: 19/491,960

(22) PCT Filed: Jul. 22, 2024
(86) PCT No.: PCT/CA2024/050967

§ 371 Date: Dec. 10, 2025

Related U.S. Application Data
(60) Provisional application No. 63/515,738, filed on Jul. 26, 2023.

(51) Int. Cl. C07H 21/02; A61K 39/00; C07B 59/00; C12N 15/113

(52) U.S. Cl. CPC C07H 21/02; A61K 39/00; C07B 59/005; C12N 15/113; A61K 2039/53; C12N 2310/14

(57) ABSTRACT

Described herein are ribonucleic acid molecules (RNAs) comprising a 5′-capping and a 3′-polyadenylation tail, wherein at least one of the 5′-capping and 3′-poly(A) tail comprises deuterium. These RNAs advantageously display improved RNA translation in living cells. Also described are methods for the synthesis of the RNAs, capping methods of RNAs, methods for polyadenylation of RNAs and methods to improve RNA translation. In embodiments the 5′-capping and/or a 3′-poly(A) tail are synthesized in the presence of deuterium such that one or more protium atoms in the capping or tail are substituted by deuterium atoms.

### CROSS REFERENCE TO RELATED APPLICATION

The present application claims priority to U.S. provisional application Ser. No. 63/515,738 filed on Jul. 26, 2023, the content of which is incorporated herein by reference in its entirety.

### FIELD OF THE INVENTION

The invention relates to the field of RNA stabilisation, and more particularly to the use of deuterium oxide (D₂O) during synthesis of RNA molecules.

### BACKGROUND OF THE INVENTION

RNA molecules are inherently unstable and prone to both non-enzymatic and enzymatic hydrolysis, which is a major issue during processing, transport, and storage. One of the most critical factors that needs to be controlled is temperature. All the mRNA vaccines and other mRNA-based therapeutics are sensitive to temperature fluctuations, which can accelerate their degradation. Therefore, there is an urgent need for thermostable mRNA therapeutics.

In International PCT Patent Publication No. WO 2022/099411, U.S. Pat. Nos. 11,566,038 and 11,780,869, Applicant was the first to successfully teach the use of deuterium to stabilize RNA molecules in aqueous solution and to use deuterium to increase their resistance of RNA molecules to thermal or enzymatic degradation.

Nevertheless, there is still a need for RNA-based therapeutics that comprises RNA molecules that display improved RNA translation in living cells.

There is still a need for mRNA molecules with a 5′-capping and/or a 3′-poly(A) tail that comprises deuterium.

There is also a need a method for the synthesis of RNA molecules that comprise a 5′-capping and/or a 3′-poly(A) tail which has been synthesized in the presence of deuterium.

The present invention addresses these needs and other needs as it will be apparent from the review of the disclosure and description of the features of the invention hereinafter.

### BRIEF SUMMARY OF THE INVENTION

According to one aspect, the invention relates to a ribonucleic acid (RNA) molecule comprising a 5′-capping and a 3′-polyadenylation tail, wherein at least one of the 5′-capping and 3′-poly(A) tail comprises deuterium.

According to another aspect, the invention relates to an ribonucleic acid (RNA) molecules, wherein said RNA molecule comprises at least one of a 5′-capping and a 3′-poly(A) tail, and wherein the at least one 5′-capping and 3′-poly(A) tail has been synthesised in the presence of deuterium.

According to another aspect, the invention relates to a method for the synthesis of an RNA molecule, wherein said RNA molecule comprises at least one of a 5′-capping and a 3′-poly(A) tail, the method comprising synthesizing the at least one 5′-capping and 3′-poly(A) tail in the presence of deuterium.

According to another aspect, the invention relates to a method for 5′-capping an RNA molecule, comprising adding a 5′ cap structure to said RNA molecule in the presence of deuterium.

According to another aspect, the invention relates to a method for polyadenylation of an RNA molecule, comprising adding a poly(A) tail to said RNA molecule in the presence of deuterium.

According to another aspect, the invention relates to an aqueous composition comprising an RNA molecule as defined herein.

According to another related aspect, the invention relates to an RNA-based therapeutic comprising an RNA molecule as defined herein.

Additional aspects, advantages and features of the present invention will become more apparent upon reading of the following non-restrictive description of preferred embodiments, which are exemplary and should not be interpreted as limiting the scope of the invention.

### BRIEF DESCRIPTION OF THE FIGURES

For the invention to be readily understood, embodiments of the invention are illustrated by way of example in the accompanying figures.

FIG. 1 show pictures comparing fluorescence in cells, 24 h after cell transfection by mRNA synthesized in light water (FIG. 1A; H-24h) or heavy water (FIG. 1B; D-24h), using transfectamine, in accordance with the first protocol of Example 1.

FIG. 2 show pictures of fluorescence in cells 6 h after cell transfection by mRNA synthesized in light water (FIG. 2A; H-6h) or heavy water (FIG. 2B; D-6h), using lipofectamine, in accordance with the first protocol of Example 1.

FIG. 3 show pictures of fluorescence in cells, 24 h after cell transfection by mRNA synthesized in light water (FIG. 3A; H-24h) or heavy water (FIG. 3B; D-24h) with sequential capping and polyadenylation, using lipofectamine, in accordance with the second protocol of Example 1.

FIG. 4 show pictures of fluorescence in cells, 24 h after cell transfection by mRNA synthesized in light water (FIG. 4A; H-24h) or heavy water (FIG. 4B; D-24h) with in vitro transcription done concomitantly with capping and polyadenylation, using lipofectamine, in accordance with the second protocol of Example 1.

FIG. 5 are graphs showing flow cytometry demonstrating expression of GFP after transfection and subsequent to phosphatase treatment of deuterated and non-deuterated mRNA, in accordance with the third protocol of Example 1 (FIG. 5A=deuterated; FIG. 5B=non-deuterated; FIG. 5C=deuterated+phosphatase; FIG. 5D=non-deuterated+phosphatase).

FIG. 6 displays the chemical structure of a uridine molecule.

FIG. 7 displays the chemical structure of a mRNA backbone showing substitution of protium to deuterium on multiple ribose.

Further details of the invention and its advantages will be apparent from the detailed description included below.

### DETAILED DESCRIPTION OF EMBODIMENTS

In the following description of the embodiments, references to the accompanying figures are illustrations of an example by which the invention may be practiced. It will be understood that other embodiments may be made without departing from the scope of the invention disclosed. Unless defined otherwise, all technical and scientific terms used herein have the same meaning as commonly understood by one of ordinary skill in the art, to which the invention belongs.

### General Overview

In International PCT Patent Publication No. WO 2022/099411, U.S. Pat. Nos. 11,566,038, 11,780,869 and US patent publication US2023/0391813 (all incorporated herein by reference in their entirety), Applicant describes, among other things, using deuterium to stabilize RNA molecules in aqueous solution and using deuterium to increase the resistance of RNA molecules to thermal or enzymatic degradation.

Applicant has now found that providing RNA molecules with a 5′-capping and/or a 3′-poly(A) tail that comprise deuterium improves translation of mRNA molecules, compared to a mRNA molecule not having a 5′-capping or 3′-poly(A) tail that comprises deuterium.

Particularly, the present disclosure describes how deuterium can be used in to increase translation of mRNAs in living cells, e.g., by synthesizing a 5′-capping and/or a 3′-poly(A) tail in the presence of deuterium.

As used herein, the term “deuterium” refers to a stable isotope of hydrogen or “heavy hydrogen” (i.e. ²H or D), ratherthan the common hydrogen-1 isotope (¹H or H, also called protium) that makes up most of the hydrogen in ambient water (H₂O). As used herein, the term “deuterium” or deuterium oxide encompass related terms and molecules such as ““deuterium oxide”, “²H₂O” and “D₂O”.

As used herein, the term “5′-capping” refers to a chemical structure at the 5′-end of RNA molecule. Typically, the 5′-capping is added by the enzyme guanyl transferase and the cap acts as a protective group from 5′ to 3′ exonuclease cleavage and it also serves as a unique identifier for recruiting protein factors for pre-mRNA splicing. The present invention encompasses various types of 5′-capping comprising one or more deuterium atom. In embodiments, the chemical structure comprises an N7-methylated guanosine linked to the first nucleotide of the RNA via a reverse 5′ to 5′ triphosphate linkage. Examples of 5′-capping encompassed by the present invention include, but is not limited to, a m7GpppG cap or a m7GpppGm cap.

As used herein, the term “3′-poly(A) tail” refers to the presence of multiple adenosine monophosphates (i.e., a stretch that only comprises adenine bases) that is found at the 3′-end of an RNA molecule. In vivo, the poly(A) tail is added to a messenger RNA (mRNA) molecule during RNA processing to increase the stability of the molecule. The present invention encompasses various types of 3′-poly(A) tail comprising one or more deuterium atom. In embodiments, the 3′-poly(A) tail comprises at least 5, or at least 10, or at least 25, or at least 50, or at least 75, or at least 100, or at least 150, or at least or at least 200, or at least 225, or at least 250 nucleotides. In embodiments, the 3′-poly(A) tail comprises about 50 to about 250 nucleotides.

RNA Molecules Comprising a 5′-Capping and/or a 3′-Polyadenylation Tail

One particular aspect of the invention concerns a ribonucleic acid (RNA) molecule comprising a 5′-capping and a 3′-polyadenylation tail, wherein at least one of said 5′-capping and 3′-poly(A) tail comprises deuterium.

Advantageously, an RNA with a 5′-capping and a 3′-polyadenylation tail comprising deuterium displays at least one of the following features, compared to an RNA molecule not having a 5′-capping or 3′-poly(A) tail comprising deuterium: i) increased structural integrity of primary and/or secondary structure; ii) increased resistance to degradation by endonucleases; and increased resistance to thermal degradation.

In embodiments, the RNA molecule consists of a mRNA molecule, and the mRNA displays greater translation compared to a mRNA molecule not having a 5′-capping or 3′-poly(A) tail comprising deuterium. In embodiments the translation is measured in living cells.

In embodiments of the invention, at least one of the 5′-capping and 3′-poly(A) tail incorporates deuterium. As used herein, the term “incorporate” or “incorporation” refers to presence of deuterium into the molecular structure of the molecule, and it encompasses integration of the deuterium isotope to the molecule via covalent, hydrogen or other type of bonding or molecular interaction.

In embodiments of the invention, at least one of the 5′-capping and 3′-poly(A) tail comprises deuterated ribonucleosides. In embodiments, at least one of the 5′-capping and 3′-poly(A) tail comprises substitution of protium atoms by deuterium atoms. In embodiments one or more protium atoms have been replaced by one or more deuterium atoms (e.g. substitution or any other mechanism by which mRNA interacts with D₂O in covalent or non-covalent fashion reducing the extent of thermal or enzymatic hydrolysis). In embodiments, at least one of the 5′-capping and 3′-poly(A) tail comprises a deuterium atom.

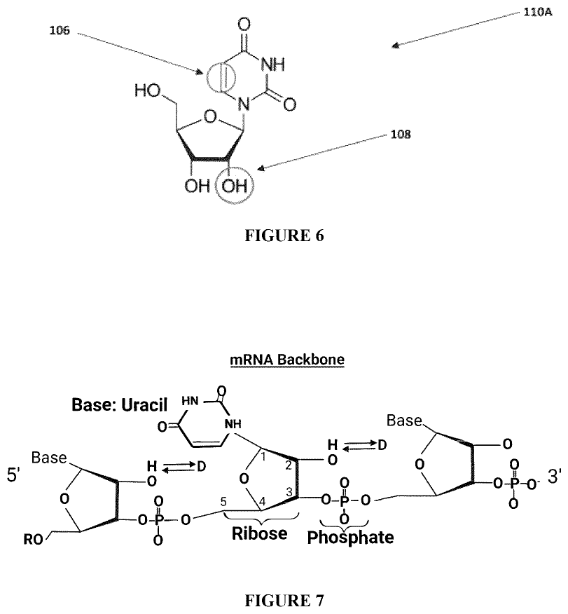

FIG. 6 depicts possible sites for deuteration in accordance with the present invention. For instance, in a uridine molecule (#110A), substitution of protium to deuterium could occur at a double bond in the uracil (5-6 position #106) or at the hydroxyl on the ribose (2′ position, #108). Likewise, FIG. 7 displays the chemical structure of a mRNA backbone showing substitution of protium to deuterium at the hydroxyl on each of the three ribose.

In embodiments, the RNA molecule sequence comprises a deuterium atom in the 2′OH-group on the ribose sugar moiety. In embodiments, the RNA molecule comprises a deuterium isotope in the RNA base itself (e.g., uracil, guanine, adenine and/or cytosine). In embodiments, the RNA molecule comprises one or more chemically modified base incorporating a deuterium isotope.

In embodiments, the RNA molecule (e.g., 5′-cap and/or or 3′-poly(A) tail) comprises deuterium atom(s) that have been incorporated in the chemical structure of the RNA during synthesis. For instance, as indicated hereinbefore, the RNA molecule can be synthesized by using deuterated ribonucleoside tri-phosphates (rNTPs).

In accordance with another embodiment, deuterium atom(s) is(are) incorporated in the RNA molecule during RNA synthesis due to the simple presence of D₂O in solution, for instance using a buffer comprising deuterium oxide. In accordance with that embodiment, deuterium incorporation into RNA molecules during RNA synthesis occurs via keto-enol tautomerization. In embodiments, the solution comprising D₂O comprises a deuterium concentration sufficient to favor thermodynamically such incorporation. In embodiments, the aqueous solution comprises a deuterium concentration of at least 5 atom % D, or at least 10 atom % D, or at least 20 atom % D, or at least 30 atom % D, or at least 40 atom % D, or at least 50 atom % D, or at least 60 atom % D, or at least 70 atom % D, or at least 80 atom % D, or at least 85 atom % D, or at least 90 atom % D, or at least 95 atom % D, or at least 96 atom % D, or at least 97 atom % D, or at least 98 atom % D, or at least 99 atom % D, or at least 99.5 atom % D, or at least 99.7 atom % D, or at least 99.9 atom % D. In embodiments, the aqueous solution comprising D₂O comprises deuterium at a concentration of about 5 atom % D to about 100 atom % D, or about 25 atom % D to about 99.9 atom % D, or about 50 atom % D to about 99.9 atom % D, or about 75 atom % D to about 99.9 atom % D, or about 85 atom % D to about 99.9 atom % D, or about 90 atom % D to about 99 atom % D, or about or about 99.7 atom % D.

In other embodiments, the deuterium atom(s) are incorporated in the RNA's chemical structure by contacting an already synthesized RNA molecule (incorporating or not deuterium) with an aqueous solution comprising D₂O. In accordance with a particular embodiment, deuterium incorporation into RNA molecules during RNA synthesis occurs via keto-enol tautomerization. In embodiments, the aqueous solution comprising D₂O comprises a deuterium concentration sufficient to favor such incorporation. In embodiments, the aqueous solution comprises a deuterium concentration of at least 5 atom % D, or at least 10 atom % D, or at least 20 atom % D, or at least 30 atom % D, or at least 40 atom % D, or at least 50 atom % D, or at least 60 atom % D, or at least 70 atom % D, or at least 80 atom % D, or at least 85 atom % D, or at least 90 atom % D, or at least 95 atom % D, or at least 96 atom % D, or at least 97 atom % D, or at least 98 atom % D, or at least 99 atom % D, or at least 99.5 atom % D, or at least 99.7 atom % D, or at least 99.9 atom % D. In embodiments, the aqueous solution comprising D₂O comprises deuterium at a concentration of about 5 atom % D to about 100 atom % D, or about 25 atom % D to about 99.9 atom % D, or about 50 atom % D to about 99.9 atom % D, or about 75 atom % D to about 99.9 atom % D, or about 85 atom % D to about 99.9 atom % D, or about 90 atom % D to about 99 atom % D, or about or about 99.7 atom % D.

The present invention further encompasses aqueous compositions including RNA molecules as defined herein. In embodiments, the aqueous composition consists of a stabilised ribonucleic acid aqueous composition comprising (i) deuterium for stabilising the RNA molecules; and/or (ii) RNA molecules that have been synthesised in the presence of deuterium (e.g., deuterium oxide). In other embodiments, the aqueous composition consists of an aqueous RNA composition comprising: (i) a first aqueous solution comprising RNA molecules, the solution comprising deuterium at a concentration sufficient for stabilising the RNA molecules; and/or (ii) a second aqueous solution comprising RNA molecules that have been synthesised in the presence of deuterium oxide (e.g. with at least one of a 5′-cap and 3′-poly(A) tail). In embodiments, the aqueous composition consists essentially of, or alternatively comprises, a stabilised ribonucleic acid aqueous composition comprising (i) deuterium for stabilising the RNA molecules; and/or (ii) RNA molecules that have been synthesized in the presence of deuterium oxide, as well as optional additional components such as RNAase inhibitor(s), enzyme(s), salts dNTPs, etc.

The present invention is not restricted to particular RNA molecules and it encompasses stabilization of various types of RNAs including, but not limited to, total RNA, messenger RNA (mRNA), silencing RNA (siRNA), small hairpin RNA (shRNA), etc. In embodiments, the RNA molecule consists of a mRNA molecule. In embodiments, the mRNA molecule is a component of a therapeutic (e.g., a vaccine such as a mRNA vaccine or else). The RNA molecule may be obtained from different source, including chemical synthesis, in vitro synthesis, in vivo synthesis, isolated or purified from different sources (e.g., cells or organisms, viruses, etc.).

### Translation Products

Another particular aspect of the invention concerns translation products obtained from translation of an RNA (e.g. mRNA molecule) as defined herein.

In one embodiment of the invention, the translation product consists of the translation product of a mRNA molecule, the mRNA molecule comprising at least one of a 5′-capping and 3′-poly(A) tail which comprises deuterium.

In embodiments, the translation product is a protein or a polypeptide.

In embodiments, RNA molecules in accordance with the invention can be integrated into living cells (e.g. in vitro, ex vivo, or in vivo) and they can be translated into functional proteins or polypeptides.

### Methods of Synthesis and Methods of Use

Additional particular aspects of the invention concern methods for making RNA molecules as defined herein (e.g., RNAs comprising a 5′-cap and/or a 3′-poly(A) tail comprising deuterium), methods for stabilising RNA molecules and methods for reducing thermal degradation of RNA molecules.

In embodiments, the method for RNA molecules as defined herein comprise synthesising the RNA molecules in an aqueous reaction media comprising D₂O. In embodiments, the method for making RNA molecules as defined herein comprises synthesising the RNA molecules by using deuterated ribonucleoside tri-phosphates (rNTPs).

A method for the synthesis of an RNA molecule, wherein said RNA molecule comprises at least one of a 5′-capping and a 3′-poly(A) tail, said method comprising synthesizing said at least one 5′-capping and 3′-poly(A) tail in the presence of deuterium.

According to one particular aspect, the invention relates to a method for the synthesis of an RNA molecule which comprises at least one of a 5′-capping and a 3′-poly(A) tail. In one embodiment the method comprises synthesizing the 5′-capping and/or 3′-poly(A) tail in the presence of deuterium.

In one embodiment the RNA molecule comprises both a 5′-capping and a 3′-poly(A) tail, and the method comprises synthesizing the 5′-capping and the 3′-poly(A) tail in the presence of deuterium.

In one embodiment, the RNA molecule is also synthesized the presence of deuterium.

In one embodiment, the RNA molecule, at least one of the 5′-capping, and the 3′-poly(A) tail are synthesized sequentially in the presence of deuterium.

In one embodiment, the RNA molecule and at least one of the 5′-capping and the 3′-poly(A) tail are ass synthesized simultaneously in the presence of deuterium.

In embodiments, the RNA molecule consists of a mRNA molecule, and the synthesis of the mRNA molecule comprises in vitro transcription (IVT).

In embodiments, the synthesizing comprises synthesis in a buffer comprising deuterium oxide. In embodiments, the buffer comprises deuterium at a concentration of about 10 atom % D to about 99.9 atom % D. In embodiments, the buffer comprises deuterium at a concentration of about 85 atom % D to about 99.9 atom % D.

Another related aspect of the present invention concerns a method for 5′-capping an RNA molecule. In one embodiment the method comprises adding a 5′ cap structure to the RNA molecule in the presence of deuterium. In embodiments, the deuterium is integrated in the 5′ cap structure during the cap adding step. In embodiments, 5′ cap structure in added during transcription. Examples of possible 5′ cap structure comprises a m7GpppG cap and a m7GpppGm cap.

Another related aspect of the present invention concerns a method for polyadenylation of an RNA molecule. In one embodiment the method comprises adding a poly(A) tail to the RNA molecule in the presence of deuterium. In embodiments, the deuterium is integrated in the poly(A) tail during polyadenylation. In embodiments, the polyadenylation comprises adding about 50 to about 250 nucleotides.

In additional an aspect, the invention proposes to carry out simultaneous in vitro transcription (IVT), 5′-capping and 3′-polyadenylation in deuterium oxide buffer. In embodiments, this improves mRNA translation in live cells compared to the same process carried out in traditional buffers free of deuterium oxide.

In additional an aspect, the invention proposes to carry out sequential IVT, 5′-capping and 3′-polyadenylation in deuterium oxide buffer. In embodiments, this improves mRNA translation in live cells compared to the same processes carried out in traditional buffers free of deuterium oxide.

In embodiments, and as explained hereinbefore, in accordance with these methods, deuterium atoms may be incorporated in the RNA molecule during RNA synthesis, and/or after RNA synthesis, due to the simple presence of D₂O in solution, for instance but not limited to via keto-enol tautomerization of the RNA molecule.

In embodiments the RNA synthesis is carried out by in vitro transcription (e.g. forward transcription) in an aqueous composition comprising deuterium.

In embodiments these methods comprise at least two consecutive steps of: (a) synthesising the RNA molecule by forward transcription (e.g. in vitro transcription) in an aqueous composition comprising deuterium; and (b) storing the synthesized RNA molecule of step (a) in an aqueous solution comprising deuterium. In embodiments, the synthesising step comprises forward transcription (e.g. in vitro transcription) with deuterated ribonucleoside tri-phosphates (rNTPs).

The invention further encompasses post-transcription modifications of RNA molecules (e.g., post synthesis enzymatic modifications). In embodiments, the RNA molecules have been linearized by phosphatases.

In accordance with the methods described herein, the presence of deuterium in the reaction media and/or in the RNA storage media provides one or more of the following benefits:
i. greater translation compared to a mRNA molecule compared to a mRNA molecule not having a 5′-capping or 3′-poly(A) tail comprising deuterium;
ii. reduction of hydrolysis or degradation of the RNA molecule by endonucleases (e.g. reduce affinity of RNA to endonucleases or else);
iii. reduction of thermal degradation (e.g. hydrolysis) of the RNA molecule, for instance reduction of degradation over 0 °C such as at 0-45 °C, or at 37 °C or a challenge at 45 °C or higher;
iv. reduction of mRNA degradation during transcription;
v. increased structural integrity of the primary and/or secondary structure of the deuterium-stabilised RNA molecule, compared to a non-stabilized RNA molecule;
vi. increased structural integrity of the tertiary and/or quaternary structure of the deuterium-stabilised RNA molecule, compared to a non-stabilized RNA molecule;
vii. increasing RNA half-life;
viii. increasing bioavailability of the RNA molecule for its substrate (i.e. ribosomes, other RNA molecules, etc.).

Accordingly, the present invention further encompasses the use of deuterium as a thermostabilizer when used as a solvent. In embodiments, deuterium is used as a thermostabilizer for RNA (e.g. mRNA, siRNA, shRNA, etc.) and its thermostabilizing activity is particularly useful for reducing hydrolysis and/or degradation of RNA molecules, including, but not limited to, during extended exposures (e.g. 1 day, or 2 days, or 3 days, or 4 days, or 5 days, or 6 days, or 7 days or more) to 37 °C, and/or during a challenge at 45 °C, or at 50 °C, or at 55 °C, or at 60 °C, or at 65 °C, or at higher temperatures. The present invention further encompasses the use of deuterium for RNA stability during renaturation process when the temperature decreases. In embodiments deuterium is used as a thermostabilizer for enzymes and/or for enzymatic activity.

### Therapeutical Applications

The RNA molecules in accordance with embodiments of the present invention may find numerous applications as research tools and therapeutics (e.g. RNA chemistry, nanofabrication, delivery systems, immunization, etc.).

Potential therapeutic applications of the RNA molecules of the invention include, but are not limited to, immunization against pathogens, cancer immunotherapies, infectious disease vaccines, allergy tolerization, protein-replacement and supplementation therapies, genome engineering and genetic reprogramming.

Accordingly, an additional aspect of the invention concerns RNA-based therapeutics comprising RNA molecules as defined herein (e.g. mRNA, siRNA, shRNA, etc.). In one embodiment, the RNA-based therapeutic comprises thermostable RNA molecules resistant to temperature fluctuations. In embodiments, the thermostable RNA molecules display resistance to thermal hydrolysis after 1 day, or 2 days, or 3 days, or 4 days, or 5 days, or 6 days, or 7 days or more of exposure to 37 °C. In embodiments, the thermostable RNA molecules display resistance to thermal hydrolysis after a challenge at 45 °C, or at 50 °C, or at 55 °C, or at 60 °C, at 65 °C. In embodiments, the above resistance to thermal hydrolysis is greater than thermal resistance of corresponding non-stabilised RNA molecules. In embodiments, the RNA molecule consists of a messenger RNA (mRNA) molecule.

In embodiments, RNA molecules as defined herein are used in the manufacture of a therapeutical product (e.g. a medicament, an active pharmaceutical ingredient and/or a vaccine) and/or for research purposes. In embodiment RNA molecules as defined herein are for administration to a subject in need thereof (e.g. for injection of the RNA to the subject). The term “subject” includes organisms in which administration of RNA molecules is desirable, and wherein mRNA processing (e.g., 5′ capping, polyadenylation and/or alternative splicing exists). The term “subject” includes domestic animals (e.g. cats, dogs, horses, pigs, cows, goats, sheep), rodents (e.g. mice or rats), rabbits, squirrels, bears, primates (e.g., chimpanzees, monkeys, gorillas, and humans), wild animals such as those living in zoos (e.g. lion, tiger, elephant, and the like), birds (e.g., chickens, turkey and other domesticated poultry), fish (e.g., salmon, trout, etc.) and transgenic species thereof. In embodiments the subject is a mammal. In embodiments the subject is a human, for instance a human patient in need of treatment.

In embodiments, RNA molecules as defined herein are used for immunization and/or for other therapeutic-related intervention(s) of a subject in need thereof (e.g., for injection of the RNA to the subject).

In embodiments, the vaccine is a mRNA vaccine. In embodiments, the vaccine is for immunization against a viral or other pathogen. In embodiments, the vaccine is a vaccine against Covid-19.

Those skilled in the art will recognize, or be able to ascertain, using no more than routine experimentation, numerous equivalents to the specific procedures, embodiments, claims, and examples described herein. Such equivalents are considered to be within the scope of this invention and covered by the claims appended hereto. The invention is further illustrated by the following examples, which should not be construed as further or specifically limiting.

### EXAMPLES

This section provides examples set out to evaluate the effect of a 5′-cap which comprises deuterium and a 3′-poly(A) tail which comprises deuterium on RNA transcription.

The present example demonstrates, among other things, that synthesis of a 5′-cap structure and/or a 3′-poly(A) tail in the presence of deuterium provide for improved in in vitro transcription (IVT) of the resulting RNA molecule.

### Example 1: RNA Synthesis and Transfection in Living Cells

### First Protocol:

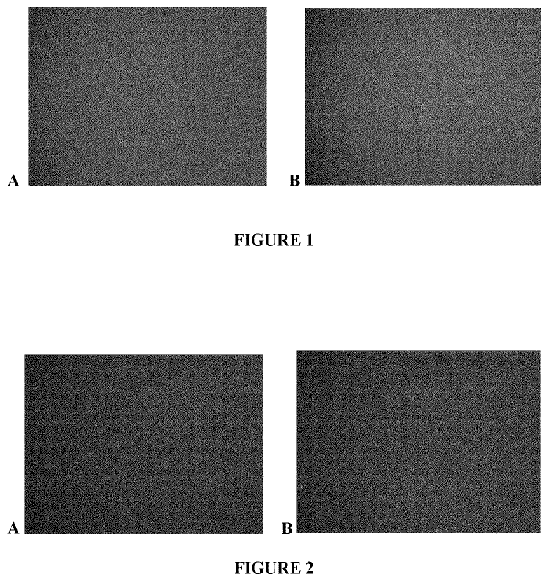

DNA isolation from Maxiprep™ was used for RNA synthesis using HiScribe® T7 High Yield RNA Synthesis Kit. RNA was purified using monarch RNA purification kit. The purified RNA was directly transfected using transfectamine reagent (FIG. 1).

Fluorescence (i.e., expression of green fluorescent protein (GFP)) was observed from 1 h-6 h post transfection: Instead of transfectamine, the next experiment used lipofectamine messenger max and the fluorescence signals were recorded starting 1h, up to 6 h and then at 22 h. The signals looked like green dots and at 22 h all the green dots were gone (FIG. 2).

### Second Protocol:

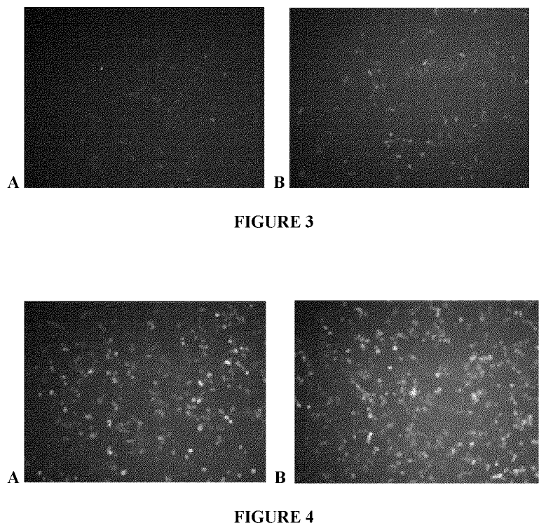

Like the first protocol, DNA isolation from Maxiprep™ was used for RNA synthesis using HiScribe® T7 High Yield RNA Synthesis Kit. This was followed by purification using monarch RNA purification kit. The RNA was capped using Vaccinia™ capping kit and purified by Monarch™ kit. The capped RNA was tailed using E. coli poly A polymerase enzyme and again purified using the same kit. RNA was then treated using Antartic™ phosphatase, followed by another step of purification. RNA was then transfected using lipofectamine messenger max reagent (FIG. 3).

### Third Protocol:

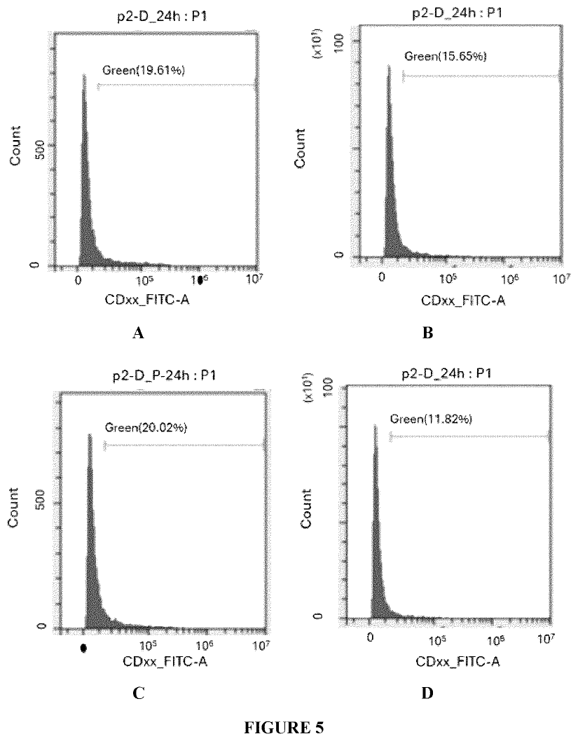

HiScribe™ T7 ARCA mRNA Kit (with tailing) was used for RNA synthesis, wherein capping and tailing occurred simultaneously with RNA transcription. Capped and tailed RNA was purified using Monarch™ kit and then treated using Antartic™ phosphatase enzyme, followed by another step of purification. RNA was then transfected using lipofectamine messenger max reagent (FIG. 4).

As can be appreciated, GFP expression was the strongest in FIG. 4, when compared to FIGS. 1-3, with an enhanced fluorescent signal in FIG. 4. Furthermore, the number of the cells expressing GFP was also higher, as shown by flow cytometry analysis (FIG. 5).

### Findings

Table 1 below summarizes the quantifications presented in FIGS. 1 to 4:

TABLE 1
Quantification results

| | | Average pixel brightness | % increase in brightness |
|---|---|---|---|
| FIG. 1 | H-24 h | 27.9 | 5.5 |
| | D-24 h | 29.4 | |
| FIG. 2 | H-6 h | 20.3 | 9.3 |
| | D-6 h | 22.2 | |
| FIG. 3 | H-24 h | 13.0 | 53.8 |
| | D-24 h | 20.1 | |
| FIG. 4 | H-24 h | 20.6 | 36.1 |
| | D-24 h | 28.1 | |

The present results suggest the following:
a. Simultaneous IVT, 5′capping and 3′polyadenylation in deuterium oxide buffer improves mRNA translation in live cells compared to the same process carried out in traditional buffers;
b. Sequential IVT, 5′capping and 3′polyadenylation in deuterium oxide buffer improves mRNA translation in live cells compared to the same processes carried out in traditional buffers;
c. Disruption of mRNA secondary structure with phosphatases does not affect the translation of deuterated mRNA in live cells, whereas the translation of nondeuterated mRNA is decreased.

Altogether the data demonstrate that 5′ capping and 3′ polyadenylation in presence of deuterium, and particularly concomitant 5′ capping and 3′ polyadenylation, increase translation efficiency of mRNA.

We can also conclude that enzymatic processing of mRNA in a deuterated environment results in better expression of proteins of interest.

Headings are included herein for reference and to aid in locating certain sections. These headings are not intended to limit the scope of the concepts described therein, and these concepts may have applicability in other sections throughout the entire specification. Thus, the present invention is not intended to be limited to the embodiments shown herein but is to be accorded the widest scope consistent with the principles and novel features disclosed herein.

As used herein, the terms, “comprises” and “comprising” are to be construed as being inclusive and open ended, and not exclusive. Specifically, when used in the specification and claims, the terms “comprises” and “comprising” and variations thereof mean the specified features, steps or components are included. These terms are not to be interpreted to exclude the presence of other features, steps or components.

The singular forms “a”, “an” and “the” include corresponding plural references unless the context clearly dictates otherwise. Thus, for example, reference to “an RNA molecule” includes one or more of such molecules and reference to “the method” includes reference to equivalent steps and methods known to those of ordinary skill in the art that could be modified or substituted for the methods described herein.

Unless otherwise indicated, all numbers expressing quantities of ingredients, reaction conditions, concentrations, properties, and so forth used in the specification and claims are to be understood as being modified in all instances by the term “about”. At the very least, each numerical parameter should at least be construed in light of the number of reported significant digits and by applying ordinary rounding techniques. Accordingly, unless indicated to the contrary, the numerical parameters set forth in the present specification and attached claims are approximations that may vary depending upon the properties sought to be obtained. Notwithstanding that the numerical ranges and parameters setting forth the broad scope of the embodiments are approximations, the numerical values set forth in the specific examples are reported as precisely as possible. Any numerical value, however, inherently contains certain errors resulting from variations in experiments, testing measurements, statistical analyses and such.

It is understood that the examples and embodiments described herein are for illustrative purposes only and that various modifications or changes in light thereof will be suggested to persons skilled in the art and are to be included within the present invention and scope of the appended claims.

1. A ribonucleic acid (RNA) molecule comprising a 5′-capping and a 3′-polyadenylation tail, wherein at least one of said 5′-capping and 3′-poly(A) tail comprises deuterium.

2. The RNA molecule of claim 1, wherein said RNA molecule comprises both a 5′-capping and a 3′-poly(A) tail comprising deuterium.

3-50. (canceled)

51. The RNA molecule of claim 1, wherein said 5′-capping comprises a m7GpppG cap or a m7GpppGm cap.

52. The RNA molecule of claim 1, wherein said RNA molecule consists of a mRNA molecule, wherein said mRNA displays greater translation compared to a mRNA molecule not having a 5′-capping or 3′-poly(A) tail comprising deuterium.

53. The RNA molecule of claim 52, wherein said RNA molecule consists of a mRNA molecule having a secondary structure, and wherein said mRNA displays greater translation after disruption said secondary structure with phosphatases compared to a mRNA molecule not having a 5′-capping or 3′-poly(A) tail comprising deuterium.

54. The RNA molecule of claim 52, wherein said greater translation is measured in living cells.

55. The RNA molecule of claim 1, wherein at least one of said 5′-capping and 3′-poly(A) tail comprises substitution of protium atoms by deuterium atoms.

56. The RNA molecule of claim 1, wherein at least one of said 5′-capping and 3′-poly(A) tail comprises a deuterium atom in the 2′OH-group on the ribose sugar moiety.

57. The RNA molecule of claim 1, wherein said RNA molecule displays at least one of the following features, compared to an RNA molecule not having a 5′-capping or 3′-poly(A) tail comprising deuterium: i) increased structural integrity of primary and/or secondary structure; ii) increased resistance to degradation by endonucleases; and iii) increased resistance to thermal degradation.

58. The RNA molecule of claim 1, wherein said RNA molecule is selected from the group consisting of total RNA, mRNA, siRNA, and shRNA.

59. The ribonucleic acid (RNA) molecule of claim 1, wherein said at least one of said 5′-capping and said 3′-poly(A) tail has been synthesised in the presence of deuterium.

60. The RNA molecule of claim 59, wherein synthesis has occurred in a buffer comprising deuterium oxide.

61. A method for the synthesis of an RNA molecule comprising a 5′-capping and a 3′-polyadenylation tail, wherein at least one of said 5′-capping and 3′-poly(A) tail comprises deuterium, said method comprising synthesizing said at least one 5′-capping and 3′-poly(A) tail in the presence of deuterium.

62. The method of claim 61, further comprising synthesizing said RNA molecule in the presence of deuterium.

63. The method of claim 62, wherein the RNA molecule, and the at least one of the 5′-capping and the 3′-poly(A) tail are synthesized simultaneously in the presence of deuterium.

64. The method of claim 61, wherein the RNA molecule consists of a mRNA molecule, and wherein synthesis of said mRNA molecule comprises in vitro transcription (IVT).

65. The method of claim 61, wherein said synthesizing comprises synthesis in a buffer comprising deuterium oxide.

66. The method of claim 61, wherein deuterium is present at a concentration of about 10 atom % D to about 99.9 atom % D.

67. The method of claim 65, wherein said buffer comprises deuterium at a concentration of about 85 atom % D to about 99.9 atom % D.

68. An RNA-based therapeutic comprising a RNA molecule as defined in claim 1.

69. The RNA-based therapeutic of claim 68, wherein said RNA-based therapeutic consists of a mRNA vaccine or a siRNA molecule.
